(19) United States
(12) Patent Application Publication
(10) Pub. No.: US 2005/0137265 A1
(43) Pub. Date: Jun. 23, 2005

(54) Rapidly dissolving metoclopramide solid oral dosage and method thereof

(76) Inventor: Eugene T. Haley, Wilmington, NC (US)

Correspondence Address: WYATT BARTON PRATT, 15 COLUMBIA AVENUE, HOPEWELL, NJ, 08525, US

(21) Appl. No.: 11/052,570

(22) Filed: Feb. 7, 2005

Related U.S. Application Data
(63) Continuation-in-part of application No. 10/401,389, filed on Mar. 31, 2003.

(51) Int. Cl. A61K 31/165

(52) U.S. Cl. 514/620

(57) ABSTRACT

A solid metoclopramide oral dosage formulation, such as a lyophilized oral dosage, dissolves within the mouth within about 60 seconds or less. This formulation is used for abating manifestations of gastroparesis in a patient, and may be taken with or without water.

### BACKGROUND OF THE INVENTION

1. Field of the Invention

The present invention provides an oral rapidly dissolving metoclopramide formulation and administration of metoclopramide of the oral immediate release formulation to abate the rapid onset of gastroparesis.

2. Brief Description of the Related Art

Gastroparesis occurs when the stomach fails to properly empty because of decreased gastric motility. Normally, the stomach contracts slowly to squeeze solid food into small compressed particles that are pushed into the small bowel. However, decreased gastric motility leads to non-movement of the food resulting in the food remaining in the stomach longer. As food lingers too long in the stomach, problems of bacterial overgrowth from the fermentation of food or food hardening into solid masses, called bezoars, may occur. Symptoms of gastroparesis may include mild or severe nausea, vomiting, abdominal discomfort and bloating, feeling of immediate fullness upon eating, and loss of appetite. Dehydration and stomach bleeding, and other medical problems may occur, as well as obstruction in the stomach such as blockage of the passage of food into the small intestine.

Metoclopramide, chemically known as 4-amino-5-chloro-2-methoxy-N-[(2-diethyl-amino)ethyl] benzamide, is used to treat gastroparesis, by stimulating stomach activity to empty the stomach. Commercially in the United States, metoclopramide is marketed under the tradename Reglan™. Reglan™ is a pharmaceutical salt of metoclopramide, metoclopramide hydrochloride, in tablet form. Complicating the matter of oral administration of metoclopramide is the fact that patients with gastroparesis often have symptoms such as vomiting and nausea as well as fullness and bloating, each of which can lead to patient discomfort with or unwillingness to swallow the available oral tablet and associated water. If vomiting takes place, the amount of metoclopramide that remains in the stomach is unknown, and the result of treatment is even less predictable.

There is a need in the art to provide a dosage form and administration of metoclopramide that overcomes the disadvantages of current commercial tablet dosages. The present invention addresses these and other needs.

### SUMMARY OF THE INVENTION

The present invention includes a solid metoclopramide oral dosage formulation comprising an oral dosage containing metoclopramide, or a pharmaceutically acceptable salt thereof, wherein the oral dosage dissolves within the mouth within a time period of from about 60 seconds or less. Preferably the solid oral dosage formulation includes a lyophilized composition.

The present invention also includes a process for formulating a lyophilized metoclopramide oral dosage having the steps of forming a solution of metoclopramide and drying by freezing in a high vacuum to form a lyophilized metoclopramide formulation This formulation is used in a method for abating manifestations of gastroparesis in a patient, with or without water.

### DETAILED DESCRIPTION OF THE PREFERRED EMBODIMENT

The present invention provides for a novel approach to treating the rapid onset of gastroparesis in a patient. The present invention includes a method for abating manifestations of gastroparesis in a patient by administering metoclopramide, including pharmaceutically acceptable salts thereof, to the patient in a manner that increases the number of patients that are able to effectively take the metoclopramide medication orally. The present invention includes a solid metoclopramide oral dosage formulation that dissolves within the mouth within a time period of from about 60 seconds or less to release the metoclopramide to the patient. Preferably the formulation of the present invention is lyophilized or freeze-dried.

Lyophilization of the metoclopramide includes freeze-drying a solution of metoclopramide into an appropriate oral dosage, such as tablet, pill, capsule, melt or other appropriate form for oral administration. The metoclopramide is placed into solution at an appropriate concentration for a final solid unit dosage. Representative liquids include water and other liquids appropriate for pharmaceutical preparations that do not degrade the metoclopramide. Once the metoclopramide is formed into solution, the metoclopramide solution is placed in unit amounts and dried by freezing in a vacuum sufficiently high for lyophilization, such as temperatures of from about −15° to about −60° C. It is to be understood that freeze drying processes can occur in other temperature ranges and at other pressure levels and that the invention contemplates the use of temperatures and reduced pressures which are effective to accomplish the objects of the invention. Additionally, other methodologies for forming an appropriate oral dosage form with proper dissolving characteristics may be used. Advantageously, the oral dosage form of the metoclopramide releases the metoclopramide in a time period of from about 60 seconds or less, such as from less than about 1 second to about 30 seconds, preferably from about 3 seconds to 15 seconds, more preferably from about 3 seconds to 10 seconds, and most preferably from about 3 seconds to about 5 seconds. Other forms of fast dissolving solid formulations may be used, such as those rapidly disintegrating formulations described in U.S. Pat. No. 4,305,502, the disclosure of which is herein incorporated by reference.

By administrating the fast dissolving solid metoclopramide formulation to the patient, the negative effects of the gastroparesis on the administration are minimized. Problematic with gastroparesis is the inability for numerous patients to effectively take oral medication, particularly with the inability or unwillingness to ingest solids or fluids while in a state of gastroparesis, including syrups or other like liquid metoclopramide formulations. With the present invention patients are able to immediately ingest the medication with or without fluids, such as water. As the lyophilized metoclopramide rapidly (defined as 60 seconds or less) dissolves in the mouth, the metoclopramide is ingested with saliva. In the most severe cases where the patient is unable to swallow fluids, the patient generally still ingests saliva which is available for ingestion with the administration of the solid oral dosage of the present invention. Unlike other oral dosage forms of metoclopramide, the rapidly dissolving oral formulation of the present invention addresses the problems of the rapid onset of gastroparesis, and as such, increases patient compliance and effectiveness. Oral dosage forms also increase patient compliance over non-oral formulations, such as nasal or other forms.

In a preferred embodiment, the present invention has similar bioequivalence of pharmacokinetics and bioavailability to known oral dosages of metoclopramide that have been proven pharmaceutically safe. However, the present invention provides increased pharmaceutical effectiveness of the present formulation residing in the fact that patients are able to take the oral metoclopramide dosage rapidly enough as to be less susceptible to the problem of vomiting and nausea. Once placed in the mouth, the mouth dissolving immediate release oral metoclopramide enters the gastrointestinal tract within about 3 to about 15 seconds. As such patients are not required to drink water to take the metoclopamide. The rapidity of the rapidly dissolving administration, and the elimination of the need to administer water, enable patients to take an effective oral metoclopramide dosage without drinking water. Patients who take the present metoclopramide formulation without water may be less likely to vomit which further increases the ability of the patient to gain the benefit of the medication, increasing its pharmaceutical effectiveness.

As many patients understand the gastroparesis consequences of certain gastroparesis causing events that they must partake in, these patients may administer the rapidly dissolving oral formulation prior to them performing the gastroparesis causing event, reducing the discomfort that the patient experiences with the rapid onset of the gastroparesis. For example, with the present invention the patient may take the rapidly dissolving formulation once seated to begin the meal, with a Tₘₐₓ occurring for example without limitation from about 55 minutes to about 65 minutes later, i.e., effectively occurring at a simultaneous time or before or during the eating process. This creates a routine and regime, particularly with older patients, that greatly increases patient compliance.

Rapid onset of the gastroparesis that occurs from the time the gastroparesis causing event occurs to the time of the gastroparesis. In the case of the intake of food, for example, gastroparesis may occur from the moment the food is not properly advanced through the stomach. Rapid onset may include, for example without limitation, times of from about forty minutes or less, such as about 0, 5, 10, 15, 20, 25, and 30 minutes, and times and ranges of times there between. Abating manifestations of the rapid onset of gastroparesis in a patient includes the elimination, reduction, mitigation or other like degrees of alleviating discomfort and/or medical complications of gastroparesis in a manner that addresses the severity and rapidity of the problem.

Gastroparesis causing events are those events that occur from a given set of circumstances. Such circumstances may include, for example without limitation, eating food, such as eating a meal including breakfast, lunch or dinner, laying down to sleep, drinking specific type of liquids, engaging in an exercise routine, engaging in an unpleasant event, such as for example attending a business meeting, school conference and the like. As such gastroparesis causing events may be known prior to the rapid onset of gastroparesis in the patient, this allows administration of metoclopramide by the patient prior to the event, either by patient self-dosing or dosing of the patient by another, such as a relative, doctor, etc.

Recurrence of gastroparesis becomes expected for certain individuals as those individuals need to repeat certain tasks in the normal course of their livelihood. In the common gastroparesis causing event of consumption of food, administration with the preparation of the eating event, such as sitting down to eat, entering a restaurant, etc., aids the person in a compliance routine that is beneficial to taking the drug. In the case of administration at bedtime, the rapid onset of gastroparesis with the act of laying down is abated, as the person laying down is aided in a regimented compliance routine.

Patients are defined herein as humans, including men, women, and children, either individuals or groups, awaiting or under medical care and/or treatment. Medical care and/or treatment includes medical care or treatment by a medical professional, such as a doctor or nurse, self-administered treatments, treatment by others, and the like. Additionally, patients include mammals other than humans, including for example without limitation, dogs, cats, and the like. In general, mammals that have previously experienced gastroparesis, or there is reason to believe that mammal would benefit from treatment for gastroparesis, are patients. Patients include a broad segment of the population, and as such, are preferably instructed by qualified persons, such as doctors, nurses, pharmacists, etc., either by prescription or other method, to take metoclopramide as taught herein. Such instruction may include, for example, taking metoclopramide in a rapidly dissolving dosage at a given time prior to eating, taking metoclopramide in a rapidly dissolving dosage with the first glass of water when eating at a restaurant, etc.

Preferably, the rapidly dissolving oral formulation comprises a mouth dissolving solid formulation that dissolves in the mouth or a rapidly dissolving formulation for the gastrointestinal tract. In addition to this gastrointestinal modality, some of the metoclopramide that dissolved in the mouth may have incidental sublingual or buccal delivery to the patient. Additionally, the oral dosage form may be immediately swallowed and dissolved within the digestive system beyond the mouth, e.g., throat, stomach, gastro-intestinal tract, etc.

Administering the rapidly dissolving formulation of metoclopramide may occur in any appropriate time period prior to the onset of gastroparesis, preferably the time period of administering the metoclopramide occurs from about thirty minutes or less prior to the gastroparesis causing event, more preferably from about twenty minutes or less prior to the gastroparesis causing event, and most preferably from about ten minutes or less, such as from about twenty minutes to about ten minutes, or from about fifteen minutes to about five minutes prior to the gastroparesis causing event. Administration of the oral immediate release formulation of the present invention also may occur after the onset of and during gastroparesis.

Metoclopramide is administered in any appropriate dosage amount that mitigates the rapid onset of gastroparesis from a gastroparesis causing event, such as preferably from about 2 mg to about 240 mg, from about 5 mg to about 160 mg, from about 5 mg to about 80 mg, from about 5 mg to about 40 mg, from about 5 mg to about 20 mg, from about 5 mg to about 10 mg, or other amounts and ranges of amounts that are effective in reducing, mitigating, or otherwise alleviating the effects of gastroparesis as determinable by one skilled in the art in light of the disclosure herein. Such amounts should be administered that are safe for the patient taking the metoclopramide dosage, and accordingly dosages in amounts of metoclopramide of from about 5 mg to about 20 mg, from about 5 mg to about 10 mg are most preferred.

The administration of the immediate release oral formulation, as taught herein, results in an abated gastroparesis event from the rapid onset of gastroparesis in the patient.

### EXAMPLE 1

### Manufacturing of Metoclopramide Oral Dosage Formulations

Metoclopramide solid oral dosage formulations are manufactured with metoclopramide hydrochloride, gelatin, mannitol, mint flavor, aspartame, and purified water. A 10% sodium hydroxide solution is used for pH adjustment to about 8.3. The metoclopramide is solubilized in a water solution and lyophilized to produce a 10 mg metoclopramide solid oral dosage formulation.

### EXAMPLE 2

The lyophilized metoclopramide oral dosage formulation of Example 1 was administered in a study of twelve subjects. Three subjects reported that the oral dosage formulation disintegrated in less than 5 seconds, three reported disintegration between 5 and 10 seconds and six reported a time period of 10 to 30 seconds. The same subjects reported that complete dispersal of the oral formulations occurred in time periods of: two subjects reported less than five seconds, two subjects reported 5 to 10 seconds, five subjects reported 10 to 30 seconds, one subject reported 50 seconds, one subject reported 55 seconds and one subject failed to report on complete dispersal.

The foregoing summary, description, and examples of the present invention are not intended to be limiting, but are only exemplary of the inventive features which are defined in the claims.

1. A metoclopramide oral dosage formulation comprising a solid oral dosage containing metoclopramide, or a pharmaceutically acceptable salt thereof, wherein the solid oral dosage dissolves within the mouth within a time period of from about 60 seconds or less.

2. The metoclopramide oral dosage formulation of claim 1, wherein the time period is from about 30 seconds or less.

3. The metoclopramide oral dosage formulation of claim 2, wherein the time period is from about 1 second to about 15 seconds.

4. The metoclopramide oral dosage formulation of claim 3, wherein the time period is from about 3 seconds to 15 seconds.

5. The metoclopramide oral dosage formulation of claim 4, wherein the time period is from about 3 seconds to 10 seconds.

6. The metoclopramide oral dosage formulation of claim 5, wherein the time period is from about 3 seconds to about 5 seconds.

7. The metoclopramide oral dosage formulation of claim 1, wherein the metoclopramide oral dosage comprises from about 40 mg of metoclopramide or less.

8. The metoclopramide oral dosage formulation of claim 7, wherein the metoclopramide oral dosage comprises about 20 mg of metoclopramide.

9. The metoclopramide oral dosage formulation of claim 1, wherein the solid oral dosage formulation comprises a lyophilized dosage form.

10. The metoclopramide oral dosage formulation of claim 1, wherein the solid oral dosage formulation comprises a dosage form selected from the group consisting of tablet, pill, capsule and melt.

11. The metoclopramide oral dosage formulation of claim 1, wherein the solid oral dosage formulation comprises a dosage form selected from the group consisting of a tablet.

12. The metoclopramide oral dosage formulation of claim 1, wherein the solid oral dosage formulation comprises a dosage form selected from the group consisting of a pill.

13. The metoclopramide oral dosage formulation of claim 1, wherein the solid oral dosage formulation comprises a dosage form selected from the group consisting of a capsule.

14. The metoclopramide oral dosage formulation of claim 1, wherein the solid oral dosage formulation comprises a dosage form selected from the group consisting of a melt.

15. A process for formulating a lyophilized metoclopramide tablet, comprising the steps of:
forming a solution of metoclopramide; and,
drying by freezing in a high vacuum to form a lyophilized metoclopramide formulation.

16. A metoclopramide lyophilized tablet product produced by the process of claim 15.

17. The metoclopramide lyophilized tablet product of claim 16, where the metoclopramide comprises a dosage of 10 mg.

18. A method for abating manifestations of gastroparesis in a patient comprising the steps of:
providing a metoclopramide oral dosage formulation of claim 1; and,
administering the oral dosage formulation effective to dissolve within the mouth within a time period of from about 60 seconds or less.

19. The method of claim 18, wherein the step of administering metoclopramide occurs without administration fluids.

20. The method of claim 19, wherein the step of administering metoclopramide occurs without administration of water.
